(19) United States
(12) Patent Application Publication
(10) Pub. No.: US 2022/0101988 A1
(43) Pub. Date: Mar. 31, 2022

(54) System And Method For Diagnostic Coding

(71) Applicant: Bruce Wayne FallHowe, Colorado Springs, CO (US)

(72) Inventor: Bruce Wayne FallHowe, Colorado Springs, CO (US)

(21) Appl. No.: 17/477,155

(22) Filed: Sep. 16, 2021

Related U.S. Application Data
(60) Provisional application No. 63/084,278, filed on Sep. 28, 2020.

(51) Int. Cl. G16H 40/20; G06F 16/245; G16H 10/60; G16H 10/40; G16H 50/20; G16H 50/70; G06Q 40/08

(52) U.S. Cl. CPC G16H 40/20; G06F 16/245; G16H 10/60; G16H 70/60; G16H 50/20; G16H 50/70; G06Q 40/08; G16H 10/40

(57) ABSTRACT

A computer implemented coding system for mandating correct medical diagnostic coding by a provider, comprises program code executable to receive preliminary medical test data associated with a patient so as to make a preliminary diagnosis. This enables a preliminary insurance code to be determined and, using a diagnostic database, to identify an outline of all medical tests that must be completed for the selected code. The system records completion of all mandated medical tests and, when complete, the treating provider makes and enters a final diagnosis. If the final diagnosis is the same as the preliminary diagnosis, the insurance code is deemed final for claims submission; otherwise, a new code is chosen and the diagnostic database is then accessed to determine the complete panoply of procedures for the new code. When the final diagnosis is the same as the preliminary diagnosis, the code is deemed correct without further audit.

### REFERENCE TO RELATED APPLICATIONS

This application is related to provisional patent application U.S. Ser. No. 63/084,278 filed Sep. 28, 2020, titled “A checklist of elements for medical billing coding that audits what must be documented in the chart” and which is incorporated in its entirety herein by reference.

### BACKGROUND OF THE INVENTION

This invention relates generally to medical billing systems and, more particularly, to a medical diagnostic coding system and method that mandates correct claims coding of a medical diagnosis and provides instant feedback to and correction by a treating provider.

Medical claim and billing systems are inherently complicated, multi-layered, redundant, and inefficient. In a typical system, a treating provider makes a preliminary diagnosis which is then matched with a billing code, such as by a “coder.” The provider's diagnosis and code may then be reviewed by a clinic or hospital coding group and, if the code is found to be incorrectly selected, the “clinic or hospital coder” may change the code. The treating provider is typically never informed of the mistake and is likely to continue the incorrect coding practice. The clinic or hospital coder may then submit a claim to a respective insurance company where the diagnosis and code are once again reviewed—this time by an “insurance coder department which is composed of coders, nurses and physician.” If the code is still incorrect, the claim will be denied. The substitute diagnosis is given by the insurance company and the diagnosis/diagnoses are paid. Once the clinic or hospital are made aware of this, their claims department writes an appeal to substantiate the original diagnostic code. The clinic or hospital appeals department documents criteria that they believe will allow the original diagnostic code to be reimbursed.

Various medical billing systems have been proposed throughout the years. Although presumably effective for their intended purposes, a common characteristic of such systems is that they still include multiple layers of claim audits, coding changes, and a lack of feedback to the treating provider.

Therefore, it would be desirable to have system and method that mandates correct diagnostic coding during a patient's first experience with a treating provider so as to minimize or even eliminate the multiple layers of claim audits, coding changes, and reimbursement modifications.

### SUMMARY OF THE INVENTION

A computer implemented coding system according to the present invention for mandating correct medical coding by a treating provider, comprises a processor for executing program code and a non-transitory computer-readable storage medium containing program code executable to perform the steps of receiving medical symptom data, exam findings and preliminary medical test data associated with the patient so that the provider can make a preliminary diagnosis of a medical condition of the patient. This enables a preliminary diagnostic code to be determined and, using a diagnostic database, to identify and generate all of the elements required for that diagnoses including a complete listing or outline of all medical tests, symptoms, physical findings on examination, consultations, procedures, and documentation that must be completed for the selected code to be submitted for reimbursement. Once the final code determined by the treating provider is corrected without needing an audit by a clinic or hospital claim staff, it may be submitted, such as via a computer network, for payment.

The system records completion of all of these auxiliary medical tests (according to the preliminary code) and, when complete, the treating provider makes and enters a final diagnosis/diagnoses. If the final diagnosis is the same as the preliminary diagnosis, the insurance code is deemed final for claims submission; otherwise, a new and more accurate code is chosen and the diagnostic database is then accessed to determine the complete panoply of procedures for said new code.

Therefore, a general object of this invention is to provide a system for diagnostic medical coding that results in a treating provider being able to determine, by using the inventive medical coding database, a final diagnosis and associated billing code without a clinic, hospital or insurance audit procedure.

Another object of this invention is to provide a diagnostic medical coding system, as aforesaid, that informs a provider of every element and procedure required of a select diagnostic code associated with a respective diagnosis.

Still another object of this invention is to provide a diagnostic medical coding system, as aforesaid, that prevents a diagnostic code to be submitted for claims processing unless and until every procedure, test and criteria associated with that code has been completed.

Yet another object of this invention is to provide a diagnostic medical coding system, as aforesaid, that provides direct and timely feedback to a treating provider regarding selection of an incorrect diagnostic code without the need and delay of a code audit.

Other objects and advantages of the present invention will become apparent from the following description taken in connection with the accompanying drawings, wherein is set forth by way of illustration and example, embodiments of this invention.

### BRIEF DESCRIPTION OF THE DRAWINGS

FIG. 1 is a block diagram illustrating a computer-implemented diagnostic coding system according to the preferred embodiment of the present invention connected to key player databases via the Internet;

FIG. 2 is a block diagram illustrating a coding database, a component of the diagnostic coding system as in FIG. 1;

FIG. 3 is a flowchart of a process executed by a processor according to the diagnostic coding system of the present invention;

FIG. 4 is a block diagram illustrating a prior art medical coding and billing system;

FIG. 5 is a block diagram illustrating a medical coding system in use with the computer-implemented diagnostic coding system according to the present invention;

FIG. 6 is a block diagram illustrating the diagnostic coding system according to the present invention in use installed on an electronic device having a processor and memory; and

FIG. 7 is a flowchart of a process as in FIG. 3 with the inclusion of a neural network having algorithms for machine learning and artificial intelligence.

### DESCRIPTION OF THE PREFERRED EMBODIMENT

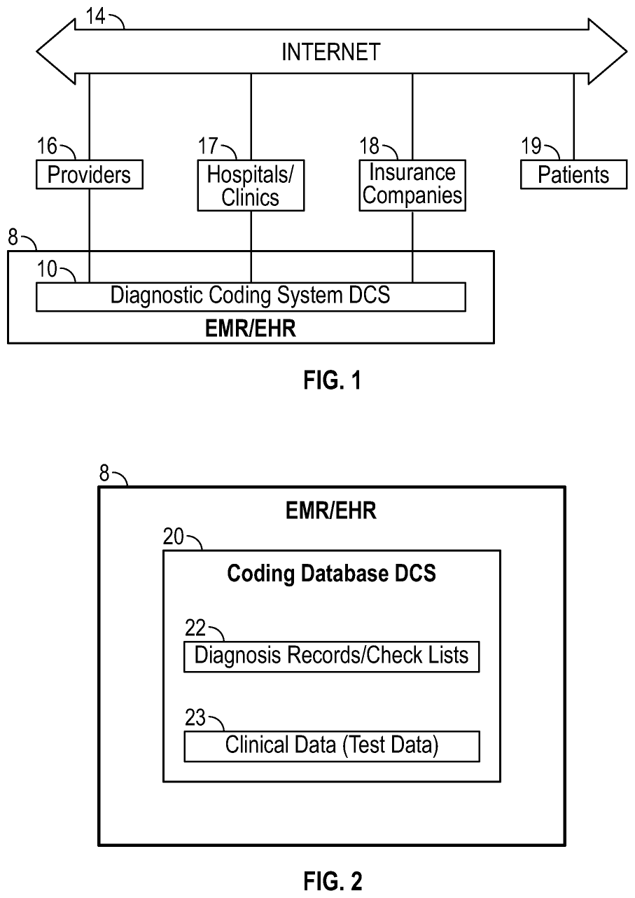

A system and method for diagnostic coding according to a preferred embodiment of the present invention will now be described with reference to FIGS. 1 to 7 of the accompanying drawings. The system 10 that mandates correct diagnostic coding includes computer software and a diagnostic coding database 20 connected to the internet and including coding executable by a processor 12.

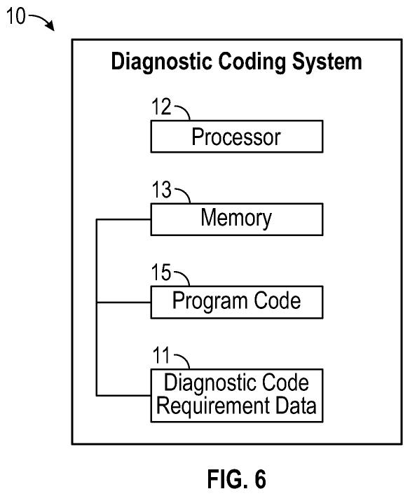

The system for diagnostic coding 10 may be stored on a non-transitory computer-readable storage medium, i.e., memory 13, for execution by a processor 12 of an electronic device connectable to a wide area network such as the Internet or local area network such as that of a clinic or hospital. Preferably, the diagnostic coding system 10 is stored on an electronic device carried by a treating provider, such as a laptop computer, a tablet, or smart phone and that is connected to the network. The diagnostic coding system 10 may include program code 15 (e.g., computer instructions) and a database that includes diagnostic code requirement data 11 (FIG. 6). As will be discussed in greater detail later, the diagnostic code requirement data 11 may include a plurality of numeric diagnostic codes each being associated with a detailed outline of medical tests and medical procedures that must be administered or satisfied in order for a respective diagnostic code to be selected. As shown particularly in FIGS. 1 and 2, it will be understood that the system for diagnostic coding 10 or at least the program code 15 may be included in the larger computer architecture of an Electronic Medical Record 8 (EMR), also known as an Electronic Health Record (EHR). An EMR are a digital version of the paper charts in the clinician's office. EMRs contain the medical and treatment history of the patients in one practice. As shown, the present system for diagnostic coding 10 may be a subset, program subroutine, or specialty program such as may be selected from within a patient's EMR 8.

In a traditional billing system, a patient may present to a provider regarding painful urination. The provider, then, may make an initial diagnosis of a “urinary tract infection” and the associated billing code is assigned. Unfortunately, however, this code may be premature until a plurality of additional testing has been completed. Currently, later lab data may contradict this coding error which must then be changed after one or more coding audits by entities other than the treating provider, e.g., by clinic, hospital or insurance company coding audits. By contrast, this preliminary error is prevented by the present invention as discussed below.

With reference to the accompanying drawings, FIG. 1 is a schematic block diagram that illustrates the electronic communications between the key parties involved in the present invention. The diagnostic coding system 10 may be implemented as computer software installed on an electronic device as described above. Each installation and operation of the diagnostic coding system 10 may include a communications module in data communication with a wide area network such as the Internet 14 or with a local computer network such as within a clinic, hospital or hospital group. This preferred connectivity enables the diagnostic coding system 10 to be used by treating provider 16, clinic, hospitals 17 or hospital coding groups 17a, insurance companies 18 or insurance company coding groups 18a, as well as directly with a consumer/patient 19_who may be in communication with the treating provider.

With reference to FIG. 2, the diagnostic coding system 10 includes a coding database 20 having a plurality of diagnosis records 22 each of which is associated with an alphanumeric code and with a comprehensive checklist or outline of symptoms, past medical history, physical examination findings, medical tests and medical procedures that must be completed or satisfied in order for the respective code to be used to describe a respective patient's diagnosis. It is understood that the coding database 20 may be stored in a non-transitory storage medium, i.e., a memory device of the electronic device upon which the diagnostic coding system 10 is installed. Further, actual clinical test data 23 may also be stored in appropriate data structures of the memory device.

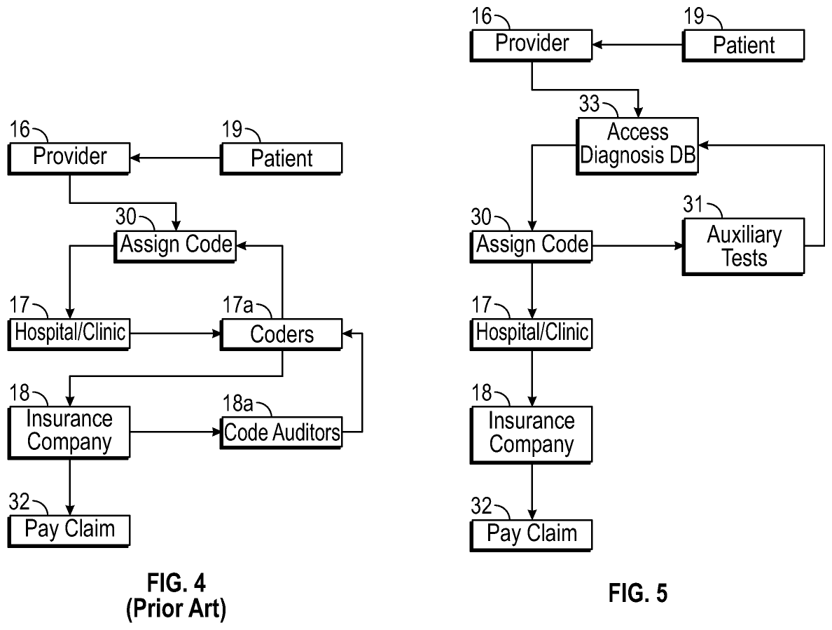

FIGS. 4 and 5 illustrate the inefficiencies of a traditional diagnostic coding scheme and the improved efficiency of a diagnostic coding method according to the present invention, respectively. With regard to a traditional diagnostic and billing system shown in FIG. 4, a treating provider 16 interacts with a medical patient 19, such as in person, via telemedicine, or the like.

Based solely on an initial examination, a patient's report, or whatever preliminary tests may be run in real time, the treating provider 16 may make a preliminary diagnosis of a medical condition and may choose a diagnosis code accordingly. But before the selected diagnostic code can be submitted as an insurance claim, the provider's preliminary diagnosis may be submitted to a clinic/hospital 17 and, more particularly, to office or hospital coding group 17a where the preliminary diagnosis is considered along with any auxiliary medical test data that may have come in to the patient's record after the preliminary diagnosis was made. The clinic or hospital coding group 17a may determine, based on a totality of the medical record, that the initial billing code is now incorrect. Similarly, the clinic or hospital coding group 17a may determine that the diagnosis is not clear and request or substitute another diagnosis code. In either instance, the clinic or hospital coders 17a may send the matter back to the treating provider 16 for reevaluation and recoding.

Once the clinic or hospital coding group 17a is satisfied with the diagnostic code, a claim may be submitted with the patient's insurance company 18 and, more particularly, to the insurance company coding auditors 18a. Once again, there is the potential that the insurance company coding auditors 18a may disagree that the claim diagnosis billed for is not supported by the documentation in the medical record. In such case, the insurance company coding department 18a will substitute another diagnosis and pay the hospital or clinic based on the substituted diagnosis.

By contrast, FIG. 5 illustrates a much more efficient and smooth process for medical diagnostic coding according to the present invention. According to the invention, a treating provider 16 interacts with a medical patient 19, such as in person, via telemedicine, or the like as illustrated in blocks 16, 19. But, then, determining a diagnosis departs from the traditional coding model. Determining a diagnostic code is generated by the coding database 20 to determine a preliminary diagnosis using data from the preliminary examination as shown at blocks, 33, 30. If the provider suspects a diagnosis different than the preliminary diagnosis, that diagnosis is entered by the provider and the diagnostic coding systems immediately presents a complete outline of tests and procedures associated with diagnosis being considered as shown at block 31. As the complete outline of indicated tests and procedure are satisfied, the process stays with the treating provider to either confirm the preliminary diagnosis (and associated code) or to modify the diagnosis and code. This methodology has the advantages of providing knowledge to the treating provider regarding the complete outline of tests and procedures required for a given diagnosis and also enables the diagnosis to be changed based on the results of the required listing of tests. This process provides the instant feedback that a provider may need to avoid making a repeated error in diagnostic coding. Further, this process assures that the final billing code selected by the treating provider is correct—making future coding audits unnecessary. Accordingly, the final diagnostic code may be submitted to a clinic or hospital billing department 17a then submits the claim to the insurance coding group 18a for payment of the claim 32 with total assurance that an audit is not necessary and that there is no need to return the coding issue back up the line.

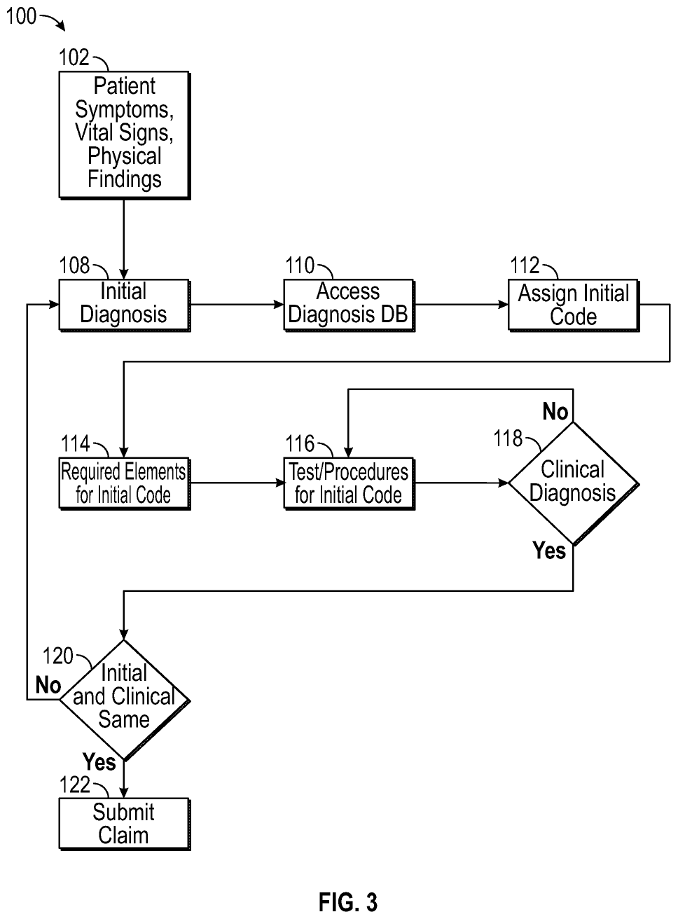

A process 100 illustrating the logic of the diagnostic coding system 10 is shown in FIG. 3. It is understood that the process 100 is preferably implemented as computer software having a plurality of program instructions (aka programming) stored in a non-transitory memory 13 and executable by a processor 12 of an electronic device used or carried by a treating provider and that is connected to the Internet 14. According to the invention, a patient presents his symptoms to a treating provider, such as in a typical office visit, or hospital setting, a telemedicine over the Internet 14, or the like. Based on the entries of the provider, the processor 12, at step 102, generates a preliminary diagnosis and proceeds to step 108.

When a preliminary diagnosis is made by a treating physician, the process proceeds to step 110 where the diagnosis database 20 is accessed, either from a position saved on the physician's electronic device or via the Internet, enabling the diagnosis to be confirmed. Accordingly, the process 100 proceeds to step 112 where a preliminary diagnostic code may be selected. The process 100 then proceeds to step 114 where an outline of all required tests and procedures that need to be administered before a respective diagnostic code may be submitted for reimbursement may be displayed and/or printed or even forwarded to persons delegated to perform the medical tests or procedures. The process 100 then passes to step 116 where the outlined tests and procedures are carried out, for instance, further bloodwork, CT scan, ultrasound, biopsies, or the like. The results of each may be saved in an associated data structure that will be referred to as “auxiliary medical test data.” Control within process 100 proceeds to step 118. It will be understood that steps 110, 112, 114 will occur almost simultaneously or may occur in an interchangeable chronological order

At step 118, the processor 12 determines if all of the tests and procedures have been completed and if treating provider has been able to determine a final or corrected diagnosis based on the auxiliary medical test data and, if so, proceeds to step 120. Otherwise, control is returned to step 116 where additional medical tests or procedures associated with the required elements for the initial code are executed. This loop between steps 116 and 118 will be repeated until all of the required medical tests and procedures associated with the preliminary diagnosis are complete and a “final diagnosis” can be made by the treating provider.

At step 120, processor 12 determines if the preliminary diagnosis is the same as the final diagnosis and, if so, the process 100 proceeds to step 122. Preferably, the processor 12 is actuated to compare the preliminary diagnostic code with a final diagnostic code to determine if one or a difference. If, the final diagnosis is now different than the preliminary diagnosis, the processor 12 will correctly determine that the diagnosis has changed due to the auxiliary test results that were generated by a totality of the tests and procedures associated with the preliminary diagnosis. In this instance, the process 100 returns control to step 108 described above so that the preliminary diagnosis may be reassessed by the treating provider and according to the diagnostic database. It is understood that this action may require additional or different procedures to be undertaken as described above. Once the preliminary diagnosis (whether original or as revised according to the auxiliary test data) is the same as the final diagnosis, control is passed to step 122.

Step 122, the final diagnostic code is deemed to be correct and without need for further audit. Accordingly, the final diagnostic code may be submitted directly to the patient's insurance company for payment through a clinic or hospital's billing department or claim processing office who then submits claims to insurance companies. Preferably, communications between a clinic or hospital and insurance company are electronic using the Internet 14 (FIG. 1).

In the last decade, the applications of machine learning (ML) algorithms have proliferated in many areas of scientific, industrial, and medical applications. In general, modern artificial intelligence (AI) systems are capable to classify with high precision very complex data. The algorithms have inspired the development of novel artificial neural network (ANN or NN) forming the basis of the field of artificial intelligence (AI). The developments have been in response to the vast amount of data, known as Big Data, which require new approaches to their processing, storage, and information extraction. Not without some limitations, AI continues to find new applications and provide fast information extraction based on algorithm embedded knowledge acquired with the use of available data in order to process newly available data: the process also known as NN training.

More particularly, the preliminary and auxiliary medical test data may be scanned and received into a natural language (NL) reader. In general, natural language processing (NLP) refers to using machine learning algorithms in relation to text and speech. More particularly, NLP may be used to create machine translations, document summaries, question answering, predictive typing, and the like. In the present case, medical documents summarizing lab work, medical advice, and other documentation may be scanned in or digitally transferred to a natural language file to be viewed by the treating provider or compared to the diagnostic code database 20 whereby the processor 12, executing programming code, is able to determine if a selected diagnostic code associated with a respective record and associated diagnosis corresponds with scanned medical test data (preliminary or auxiliary) and diagnosis (initial or final).

As indicated, the step of determining a respective record associated with a diagnosis may include providing a neural network (NN) that is in data communication with the diagnostic coding database 20 that includes algorithms configured for machine learning (ML) and artificial intelligence (AI) operable, either with electronics or programming code, to determine if received medical test data is indicative of a selected record associated with a preliminary diagnosis or respective record associated with a final diagnosis. If a final diagnosis is the same as the preliminary diagnosis (and of course the codes would be the same), then the processor may determine that an insurance claim may be made and should be paid accordingly.

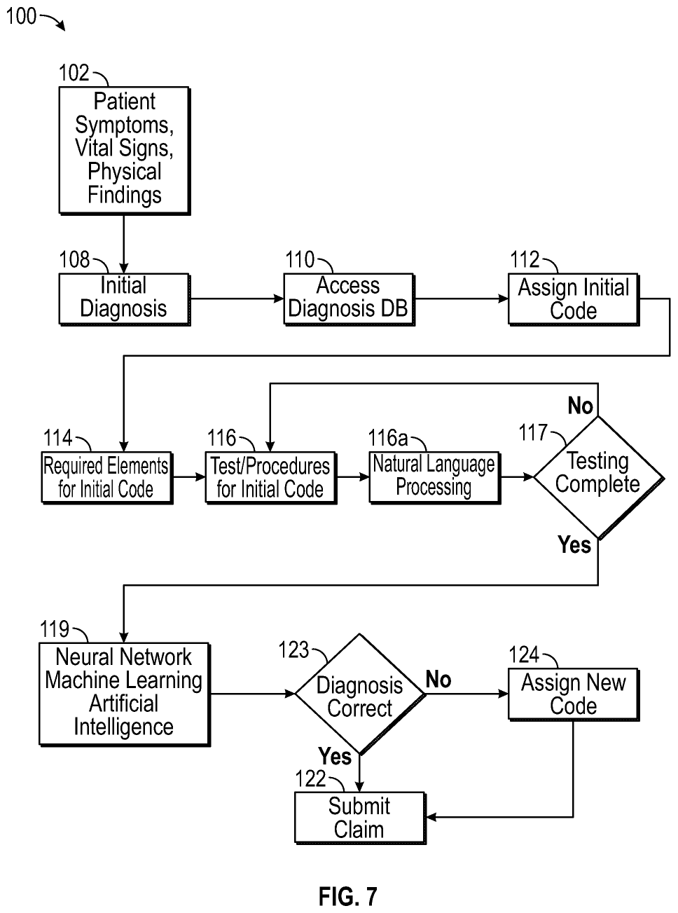

In an embodiment that includes a neural network configured to use ML/AI, FIG. 7 is a flowchart illustrating the process 100 more specifically. Only the steps that are changed from FIG. 3 will be described below. Starting at step 116, the tests and procedures associated with the preliminary diagnosis (and its code) are carried out and the results may undergo natural language processing at step 116a and with processed data being recorded in a natural language file capable of being read by the treating provider or by the ML/AI component. Then at step 117, the processor 12 determines if medical testing is complete and, if not, the process 100 loops to step 116. Otherwise, the process 100 proceeds to step 119.

At step 119, the totality of auxiliary medical test data may be received by the neural network and the ML/AI engine which has been trained according to historical medical data and, specifically, according to the diagnostic coding database 20. The process 100 proceeds to step 123 where the processor 14, via programming, determines if the diagnosis of the treating provider (and associated code) is correct according to the medical data evaluated by the ML/AI and, if so, the process proceeds to step 122 where a claim is made to an insurance company for payment. Otherwise (if the diagnosis is not correct according to the medical data evaluated by the ML/AI), the process 100 proceeds to step 124 where the ML/AI is configured to determine a code/diagnosis that matches the totality of medical test data. The changed code is deemed correct and the process 100 proceeds to step 122 where a claim may be submitted.

In an embodiment, the neural network with ML is not included to confirm a code derived from the diagnostic coding database 20, but rather is provided in place of it. In other words, the NN may not be in data communication with the diagnostic coding database 20 as described above but, rather, may be operable to determine if received medical test data is indicative of the diagnosis and, if not, to determine a diagnosis consistent with the submitted medical test data.

In use, the system and method for correct medical diagnostic coding 10 enables a treating provider to determine a correct final diagnostic code associated with a final diagnosis of a patient without a need for the clinic, hospital or insurance company audits. More particularly, the present system combines a medical billing system with a diagnostic database having a plurality of records each providing a detailed outline disclosing all of the medical tests and medical procedures that must be completed and satisfied in order for a medical diagnostic code to be reimbursed. Further, the present system provides a treating provider with instant feedback regarding choosing a correct preliminary diagnosis and associated preliminary diagnostic code as well as latitude to revise said associated preliminary diagnostic code once all medical tests and procedures associated with a preliminary diagnosis have been performed and evaluated. The present system mandates that a diagnostic code will only be submitted to a patient's insurance company if all medically required tests and procedures have been completed and so long as the treating provider confirms that a final diagnosis matches a preliminary diagnosis and all required tests and procedures have been completed. In an embodiment, the step of determining a correct coding of a diagnosis includes a neural network configured for machine learning and artificial intelligence to which a totality of the auxiliary medical test data may be submitted and evaluated.

It is understood that while certain forms of this invention have been illustrated and described, it is not limited thereto except insofar as such limitations are included in the following claims and allowable functional equivalents thereof.

1. A computer implemented system for mandating correct medical coding by a treating provider of a patient, said system comprising:
a non-transitory computer-readable storage medium containing program code and data structures;
a diagnostic coding database having a plurality of records each including at least a medical diagnosis and a diagnostic requirement portfolio associated with a respective record;
a processor in data communication with said computer-readable storage medium and operative to execute said program code to perform the steps of:
receiving medical symptom data from the patient;
receiving preliminary medical test data associated with the patient;
receiving a preliminary diagnosis from the treating provider of a medical condition of the patient based on said received medical symptom data and said preliminary medical test data;
determining from said diagnostic coding database a respective record associated with said preliminary diagnosis.

2. The system as in claim 1, wherein each diagnostic requirement portfolio includes an outline of predetermined medical conditions that must be determined before a code associated with said preliminary diagnosis can be deemed final.

3. The system as in claim 2, wherein said processor is configured to execute said program code to perform the steps of:
receiving auxiliary medical test data associated with the patient according to said diagnostic requirement portfolio associated with said preliminary diagnosis;
repeatedly receiving said auxiliary medical test data associated with the patient according to said diagnostic requirement portfolio until all elements of said diagnostic requirement portfolio associated with said preliminary diagnosis have been completed.

4. The system as in claim 3, wherein said processor is configured to execute said program code to perform the steps of:
receiving a final diagnosis from the treating provider of the medical condition of the patient based on said received auxiliary medical test data; and
determining if said final diagnosis is the same as said preliminary diagnosis and, if so, determining that a final diagnosis is equal to said preliminary diagnosis.

5. The system as in claim 4, wherein said processor is configured to execute said program code to perform the steps of:
using the diagnostic coding database to determine an insurance code associated with said preliminary diagnosis;
using the diagnostic coding database to determine an insurance code associated with said final diagnosis.

6. The system as in claim 5, wherein said processor is configured to execute said program code to perform the steps of:
on a display screen in data communication with said processor, displaying said diagnostic requirement portfolio associated with said preliminary diagnosis;
displaying on a display screen said preliminary diagnosis and the insurance code associated with said preliminary diagnosis; and
displaying on said display screen said final diagnosis and the insurance code associated with said final diagnosis.

7. The system as in claim 4, wherein said processor is configured to execute said program code to perform the steps of:
if said final diagnosis is not the same as said preliminary diagnosis, receiving a new preliminary diagnosis from the treating provider regarding the medical condition of the patient based on said auxiliary medical test data;
determining from said diagnostic coding database a respective record associated with said new preliminary diagnosis.

8. The system as in claim 5, wherein said processor is configured to execute said program code to perform the step of communicating said insurance code associated with said final diagnosis to a third-party coder via a wide area network.

9. The system as in claim 8, wherein said third party coder is an insurance company.

10. The system as in claim 8, wherein said third party coder is a clinic or hospital.

11. A computer implemented system for mandating correct medical coding by a treating provider of a patient comprises a processor for executing program code and a non-transitory computer-readable storage medium containing program code executable to perform the steps of:
receiving medical symptom data from the patient;
receiving preliminary medical test data associated with the patient;
receiving a preliminary diagnosis from the treating provider of a medical condition of the patient based on said received medical symptom data and said preliminary medical test data;
providing a diagnostic coding database having a plurality of records each including at least a medical diagnosis and a diagnostic requirement portfolio associated with a respective record;
using said diagnostic coding database, determining a respective record associated with said preliminary diagnosis, wherein said diagnostic coding database has a plurality of records each including at least a medical diagnosis and a diagnostic requirement portfolio associated with a respective record, wherein each diagnostic requirement portfolio includes an outline of predetermined medical conditions associated with said medical diagnosis, respectively; and
receiving auxiliary medical test data associated with the patient according to said diagnostic requirement portfolio associated with said preliminary diagnosis;
repeatedly receiving said auxiliary medical test data associated with the patient according to said diagnostic requirement portfolio until all elements of said diagnostic requirement portfolio associated with said preliminary diagnosis have been completed.

12. The method as in claim 11, wherein said determining a respective record associated with said preliminary diagnosis includes providing a neural network (NN) in data communication with said diagnostic coding database having algorithms configured for machine learning (ML) and artificial intelligence (AI) operable to determine if a totality of said received auxiliary medical test data is indicative of said respective record associated with said preliminary diagnosis and, if so, recommending making a claim for payment.

13. The method as in claim 12, wherein said neural network (NN) is operable select a different record that is indicated by said totality of said received auxiliary medical test data.

14. The method as in claim 13, wherein said ML/AI algorithms are trained using standard test waveforms and recorded historical data indicative of said plurality of records each including at least a medical diagnosis and a diagnostic requirement portfolio associated with a respective record.

15. The method as in claim 11, wherein said receiving auxiliary medical test data includes receiving a natural language file generated by a natural language (NL) reader configured to receive said auxiliary medical test data.

16. The system as in claim 11, wherein said processor is configured to execute said program code to perform the steps of:
receiving a final diagnosis from the treating provider of the medical condition of the patient based on a totality of said received auxiliary medical test data; and
determining if said final diagnosis is the same as said preliminary diagnosis and, if so, determining that said final diagnosis is unchanged from said preliminary diagnosis.

17. The system as in claim 12, wherein said processor is configured to execute said program code to perform the steps of:
using the diagnostic coding database to determine an insurance code associated with said preliminary diagnosis;
using the diagnostic coding database to determine an insurance code associated with said final diagnosis.

18. The system as in claim 13, wherein said processor is configured to execute said program code to perform the steps of:
on a display screen in data communication with said processor, displaying said diagnostic requirement portfolio associated with said preliminary diagnosis;
displaying on a display screen said preliminary diagnosis and the insurance code associated with said preliminary diagnosis; and
displaying on said display screen said final diagnosis and the insurance code associated with said final diagnosis.

19. The system as in claim 16, wherein said processor is configured to execute said program code to perform the steps of:
if said final diagnosis is not the same as said preliminary diagnosis, receiving a new preliminary diagnosis from the treating provider regarding the medical condition of the patient based on said auxiliary medical test data;
determining from said diagnostic coding database a respective record associated with said new preliminary diagnosis.

20. The system as in claim 13, wherein said processor is configured to execute said program code to perform the step of communicating said insurance code associated with said final diagnosis to a third-party coder via a wide area network.

21. The system as in claim 16, wherein said third-party coder is an insurance company.

22. The system as in claim 16, wherein said third party coder is a clinic or hospital.
